(19) United States
(12) Patent Application Publication
(10) Pub. No.: US 2012/0238813 A1
(43) Pub. Date: Sep. 20, 2012

(54) PROPULSION ASSEMBLY FOR ENDOSCOPE

(76) Inventors: Tsuyoshi Ashida, Kanagawa (JP); Takayuki Nakamura, Kanagawa (JP); Shinichi Yamakawa, Kanagawa (JP); Yasunori Ohta, Kanagawa (JP)

(21) Appl. No.: 13/406,616

(22) Filed: Feb. 28, 2012

(30) Foreign Application Priority Data
Mar. 15, 2011 (JP) 2011-056343

(51) Int. Cl. A61B 1/00

(52) U.S. Cl. 600/114

(57) ABSTRACT

A propulsion assembly is for an endoscope having an elongated tube for entry in a tube of a body cavity. The propulsion assembly includes endless belts for contacting a surface of the body cavity, for propulsion by turning around in an axial direction of the elongated tube. A support sleeve supports the endless belts movably. A distal cover frame is secured to a distal side of the support sleeve. An idler roller is contained in the distal cover frame, engaged with a distal end of the endless belts, for regulating the endless belts in the axial direction. A proximal cover frame is secured to a proximal side of the support sleeve. A driving roller is contained in the proximal cover frame, engaged with a proximal end of the endless belts, for regulating the endless belts in the axial direction. The driving roller drives the endless belts.

### BACKGROUND OF THE INVENTION

1. Field of the Invention

The present invention relates to a propulsion assembly for an endoscope. More particularly, the present invention relates to a propulsion assembly for an endoscope, in which physical stress to a patient's body can be reduced effectively during imaging.

2. Description Related to the Prior Art

An endoscope includes a steering device and an elongated tube for entry in a tube of a body cavity of a patient. The steering device steers a head assembly in a direction as desired. Portions of a large intestine where entry of the endoscope is locally very difficult include a sigmoid colon and transverse colon in the body cavity. Manipulation of the endoscope is a difficult process, because the large intestine is a tortuous organ in a human body, and some body parts are very changeable in the position in the body. If a doctor is insufficiently skilled in the manipulation, physical load to the body will be very large.

U.S. Pat. Nos. 6,971,990 and 7,736,300 (corresponding to JP-A 2009-513250) disclose a propulsion assembly for propelling an endoscope in an axial direction in a body cavity. The propulsion assembly includes an endless track device in a toroidal shape with an annular surface, for advancing the endoscope by turning the endless track device.

However, there is a problem in that the propulsion assembly has a large diameter because of an internally disposed driving roller for driving the endless track device. Physical stress to the patient's body during entry of the propulsion assembly is considerably large.

### SUMMARY OF THE INVENTION

In view of the foregoing problems, an object of the present invention is to provide a propulsion assembly for an endoscope, in which physical stress to a patient's body can be reduced effectively during imaging.

In order to achieve the above and other objects and advantages of this invention, a propulsion assembly for an endoscope having a section of an elongated tube for entry in a tube of a body cavity is provided. There is an endless track device for contacting a surface of the body cavity, and for propulsion by turning around in an axial direction of the elongated tube. A support sleeve receives the elongated tube therein, and supports the endless track device movably. A distal cover frame has a first through opening, for covering a distal side of the support sleeve in receiving the elongated tube in the first through opening. A first regulating portion is contained in the distal cover frame, for contacting a distal end of the endless track device, to regulate the endless track device in the axial direction. A proximal cover frame has a second through opening, for covering a proximal side of the support sleeve in receiving the elongated tube in the second through opening. A second regulating portion is contained in the proximal cover frame, for contacting a proximal end of the endless track device, to regulate the endless track device in the axial direction. At least one of the first and second regulating portions is a driving device, rotated by a drive source, for driving the endless track device.

The endless track device includes an upper run, disposed outside the support sleeve to extend in the axial direction, for contacting the surface of the body cavity. A lower run is disposed inside the support sleeve to extend in the axial direction, and opposed to a peripheral surface of the elongated tube.

The lower run movably contacts the peripheral surface of the elongated tube.

The second regulating portion is the driving roller.

The first regulating portion is an idler roller for rotating upon movement of the endless track device.

The endless track device includes plural endless belts arranged around the elongated tube to extend respectively in the axial direction.

The support sleeve includes a distal sleeve part disposed on a proximal side from the first regulating portion. A proximal sleeve part is disposed between the distal sleeve part and the second regulating portion. A compression spring device is disposed between the proximal and distal sleeve parts, for biasing the distal sleeve part toward the first regulating portion, and biasing the proximal sleeve part toward the second regulating portion.

Furthermore, a rotatable control wire is disposed to extend along the elongated tube. An input gear is secured to a distal end of the control wire. A spur gear ring is supported on the proximal cover frame, disposed around the elongated tube, and caused to rotate by mesh with the input gear. Worm gear teeth are formed at a distal end of the spur gear ring. The driving roller has teeth and rotates in mesh with the worm gear teeth.

Furthermore, a coupling device secures the support sleeve to at least one of the proximal and distal cover frames.

The coupling device includes an engaging projection, formed to project from a portion of the support sleeve between the endless belts, for retention inside the proximal or distal cover frame.

Furthermore, a distal support roller is secured to the support sleeve in a rotatable manner, opposed to the first regulating portion, for supporting each one of the endless belts movably. A proximal support roller is secured to the support sleeve in a rotatable manner, opposed to the second regulating portion, for supporting each one of the endless belts movably.

The distal support roller includes a first rotatable roll disposed on an inner wall of the support sleeve in a rotatable manner. A second rotatable roll is disposed on an outer wall of the support sleeve in a rotatable manner. The first regulating portion projects between the first and second rotatable rolls in a proximal direction.

The first regulating portion is a pad portion formed to project from an inner surface of the distal cover frame toward the support sleeve.

Furthermore, a first support surface is formed at a distal end of the support sleeve, curved arcuately, opposed to the first regulating portion, for supporting each one of the endless belts movably. A second support surface is formed at a proximal end of the support sleeve, curved arcuately, opposed to the second regulating portion, for supporting each one of the endless belts movably.

Accordingly, physical stress to a patient's body can be reduced effectively during imaging, because a diameter of the propulsion assembly can be small owing to the arrangement of the proximal and distal cover frames and the driving roller in the axial direction.

### BRIEF DESCRIPTION OF THE DRAWINGS

The above objects and advantages of the present invention will become more apparent from the following detailed description when read in connection with the accompanying drawings, in which:

FIG. 1 is a plan illustrating an endoscope system;

FIG. 2 is a perspective view illustrating a head assembly of an endoscope and a propulsion assembly;

FIG. 3 is a vertical section illustrating the propulsion assembly;

FIG. 4 is an exploded perspective view illustrating the propulsion assembly;

FIG. 5 is an exploded perspective view illustrating a distal cover frame;

FIG. 6 is an exploded perspective view illustrating a proximal cover frame;

FIG. 7 is a vertical section illustrating one preferred propulsion assembly having two sleeve parts;

FIG. 8 is a vertical section illustrating another preferred propulsion assembly having a stationary pad portion;

FIG. 9 is a vertical section illustrating still another preferred propulsion assembly having a support sleeve with rotatable rolls for an endless belt;

FIG. 10 is a vertical section illustrating another preferred propulsion assembly having a support sleeve with curved support surfaces;

FIG. 11 is a vertical section illustrating a further preferred propulsion assembly having a support sleeve with engaging projections.

### DETAILED DESCRIPTION OF THE PREFERRED EMBODIMENT(S) OF THE PRESENT INVENTION

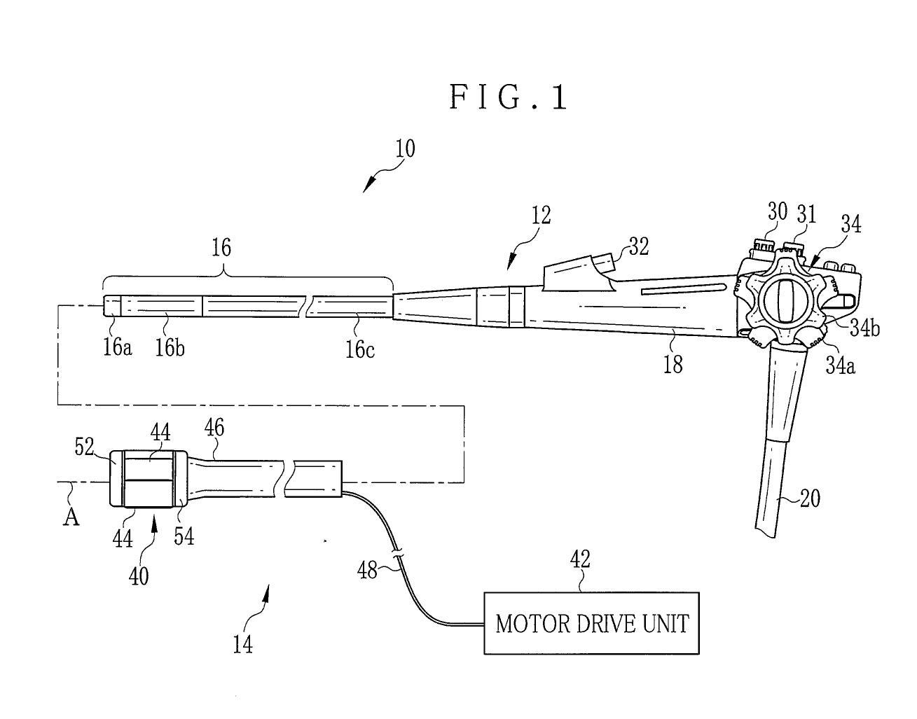

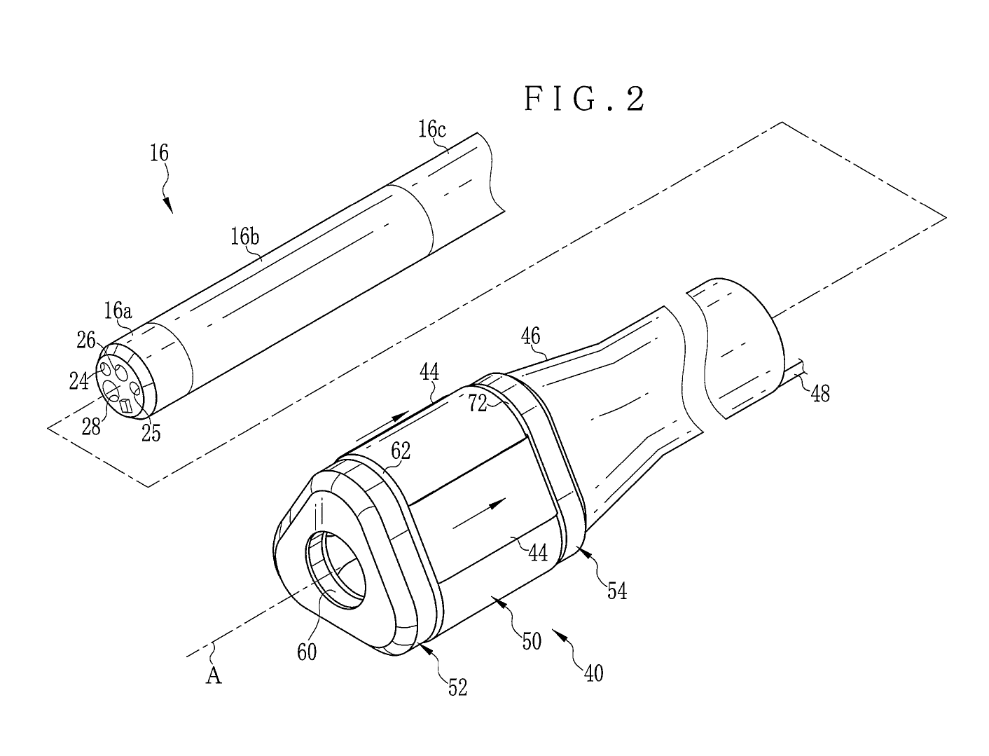

In FIGS. 1 and 2, an endoscope system 10 includes an electronic endoscope 12 and a propulsion assembly 14 for the endoscope 12. The endoscope 12 includes a section of an elongated tube 16 or guide tube, and a handle device 18. The elongated tube 16 is for entry in a tube of a body cavity, for example, large intestine. The handle device 18 is disposed at a proximal end of the elongated tube 16. A universal cable 20 extends from the handle device 18. External apparatuses are connected to the endoscope 12 by the universal cable 20, including a processing apparatus, light source apparatus, and fluid supply source (all not shown).

The elongated tube 16 includes a head assembly 16a, a steering device 16b and a flexible device 16c arranged in a proximal direction. The head assembly 16a includes a lighting window 22, end nozzles 24 and 25, and a distal instrument opening 26. The lighting window 22 emits light from the light source apparatus to an object of interest in the body cavity. The end nozzles 24 and 25 eject air and water from the fluid supply source toward the imaging window. The distal instrument opening 26 is an end opening of an instrument channel 32. A tip of an electrocautery device or other instruments appears through the distal instrument opening 26.

An imaging window 28 is formed in the head assembly 16a for receiving object light from an object in a body cavity. A lens system and an image sensor are disposed behind the imaging window 28. Examples of the image sensor are CCD, CMOS and other solid state imaging devices. The image sensor is connected to a processing apparatus (not shown) by a signal cable which is disposed to extend through the elongated tube 16, the handle device 18 and the universal cable 20. The processing apparatus drives the image sensor to form an image of the object, and drives a monitor display panel (not shown) to display the image.

The steering device 16b is flexible and is connected to the handle device 18 by a wire or the like. The steering device 16b is steered up or down or to the right or left by operation of the handle device 18. Thus, the head assembly 16a can be bent in a desired direction. The flexible device 16c has as large a length as several meters for reach of the head assembly 16a flexibly to an object of interest in the body cavity.

The handle device 18 includes fluid supply buttons 30 and 31 and the instrument channel 32. The fluid supply buttons 30 and 31 are operable for supply of air and water through the end nozzle 24. A steering control unit 34 is incorporated in the handle device 18, and includes two steering wheels 34a and 34b. When the steering wheel 34a is rotated, the steering device 16b is steered up and down. When the steering wheel 34b is rotated, the steering device 16b is steered to the right and left.

The propulsion assembly 14 is mounted on the endoscope 12, and moves the elongated tube 16 of the endoscope 12 forwards and backwards in a body cavity. The propulsion assembly 14 includes an endless track device 40 or propulsion device, and a motor drive unit 42. The endless track device 40 is mounted on the elongated tube 16 and enters the body cavity. The motor drive unit 42 is a drive source disposed externally, and controls the endless track device 40.

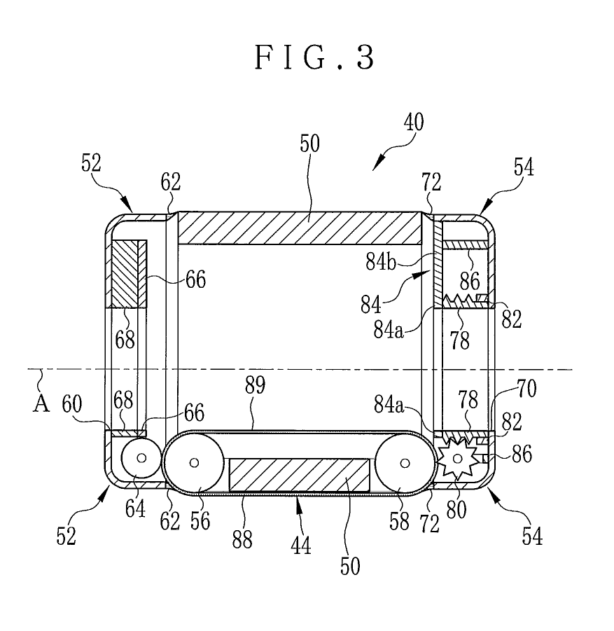

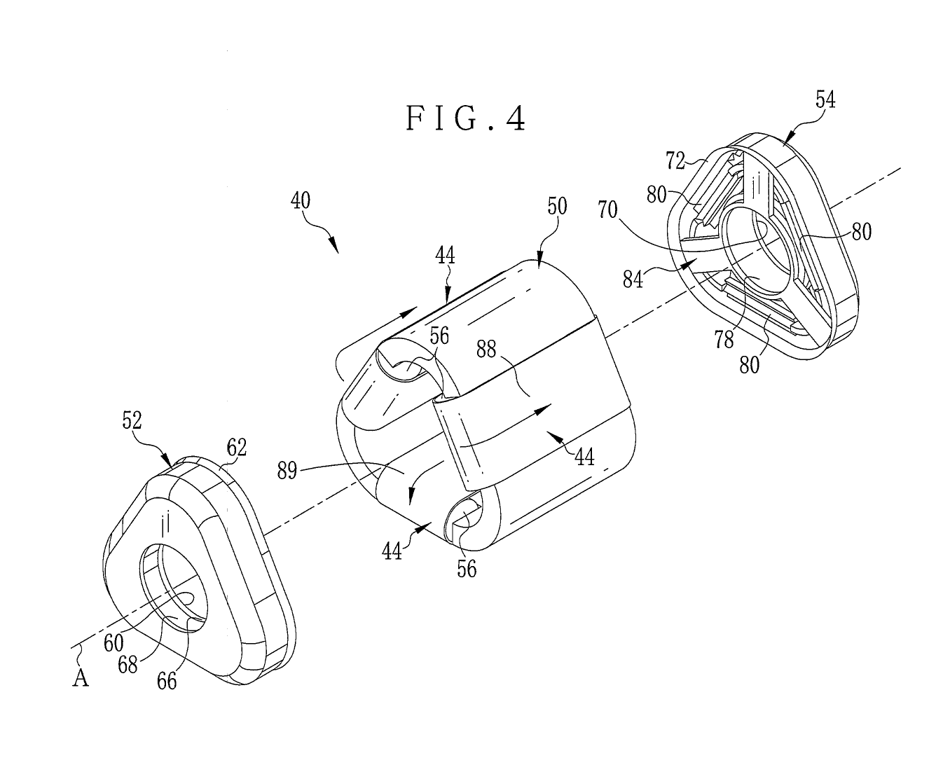

The endless track device 40 includes three endless belts 44 formed from flexible biocompatible plastic material, such as polyvinyl chloride resin, polyamide resin, fluorocarbon resin, polyurethane resin and the like. The endless belts 44 are arranged in a rotationally symmetrical manner at an angular interval of 120 degrees about the axial direction A of the elongated tube 16. The endless belts 44 are turned in the axial direction A. In the propulsion assembly 14, an upper belt run 88 (See FIGS. 3 and 4) generates force of propulsion of the elongated tube 16 by driving the endless belts 44.

An overtube 46 is connected to a proximal end of the endless track device 40, and formed from flexible biocompatible plastic material, such as polyvinyl chloride resin, polyamide resin, fluorocarbon resin, polyurethane resin and the like. A control wire 48 is entered through the overtube 46 for applying rotational force to the endless belts 44. A distal end of the control wire 48 is connected to the endless track device 40. A proximal end of the control wire 48 is connected to the motor drive unit 42.

The motor drive unit 42 includes a motor and an input interface (not shown) with buttons. The motor rotates the control wire 48. The input interface is used to select one of rotational directions of the motor and adjust its rotational speed. Thus, a direction and speed of the propulsion of the elongated tube 16 can be changed by operating the input interface, because the turn around of the endless belts 44 is controlled.

In FIGS. 3 and 4, the endless track device 40 includes a support sleeve 50, a distal cover frame 52 or cover, and a proximal cover frame 54 or cover. The support sleeve 50 supports the endless belts 44. The distal cover frame 52 is disposed on a distal side from the support sleeve 50. The proximal cover frame 54 is disposed on a proximal side from the support sleeve 50. Those are mounted on the elongated tube 16 in a sequence of the proximal cover frame 54, the support sleeve 50 and the distal cover frame 52.

The support sleeve 50 is in a shape of a triangular prism, and disposed around the elongated tube 16. A distal support roller 56 and a proximal support roller 58 are supported at respectively distal and proximal ends of the support sleeve 50 in a rotatable manner. Three of the distal support roller 56 are arranged in a rotationally symmetrical manner at an angular interval of 120 degrees about the axial direction A of the elongated tube 16. Three of the proximal support roller 58 are arranged in a similar manner. Each of the endless belts 44 extends and is supported by the proximal and distal support rollers 56 and 58 on a path of the support sleeve 50, and is turned around by rotation of the proximal and distal support rollers 56 and 58.

A size and a shape of the support sleeve 50 is predetermined so that a lower belt run 89 slightly contacts the periphery of the elongated tube 16 upon turning of the endless belts 44. The support sleeve 50 is kept from shifting transversely to the axial direction A of the elongated tube 16. It is possible to reduce an outer diameter of the propulsion assembly 14 because no mechanical element is disposed between the lower belt run 89 and the periphery of the elongated tube 16.

The distal cover frame 52 is in a shape for closing on a distal side of the support sleeve 50. A through opening 60 is formed in the distal cover frame 52, receives entry of the elongated tube 16 and is mounted thereon. A cover flange 62 or wiper flange is disposed on a proximal edge of the distal cover frame 52, and formed from flexible material. When the distal cover frame 52 is mounted on the elongated tube 16, the cover flange 62 closes a clearance space between the distal cover frame 52, the support sleeve 50 and the endless belts 44. This is effective in preventing entry of foreign material between the support sleeve 50 and the periphery of the elongated tube 16. Also, the cover flange 62 prevents entanglement of tissue of an inner wall of a body cavity in the course of turning the endless belts 44, such as tissue of intestines. The distal cover frame 52 prevents the support sleeve 50 from shifting in the distal direction.

In FIG. 5, an idler roller 64 as a first regulating portion is contained in the distal cover frame 52. A roller holder 66 of a triangular plate supports the idler roller 64 in a rotatable manner. Three of the idler roller 64 are arranged on the roller holder 66 at an interval of 120 degrees about the axial direction A of the elongated tube 16. There is a spacer 68 with which the roller holder 66 is fixed to a proximal surface of the distal cover frame 52. The idler roller 64 is engaged with the endless belts 44 by positioning the distal cover frame 52 and is caused to rotate by turning of the endless belts 44.

The support sleeve 50 can be prevented from shifting in the distal direction, because the distal cover frame 52 keeps the idler roller 64 engaged with the endless belts 44. Note that the distal cover frame 52 is also effective in preventing the support sleeve 50 from shifting in the distal direction. It is possible to omit the idler roller 64. Lack of the idler roller 64, although force for regulating the support sleeve 50 is smaller, can reduce a length of the propulsion assembly 14 in the axial direction.

In FIGS. 3 and 4, a through opening 70 is formed in the proximal cover frame 54. The proximal cover frame 54 is in a shape for closing. The elongated tube 16 is combined with the proximal cover frame 54 by entry of the elongated tube 16 in the through opening 70. A cover flange 72 or wiper flange is disposed at a distal edge of the proximal cover frame 54, and is formed from flexible material, and when the proximal cover frame 54 is mounted on the elongated tube 16, closes a clearance space between the proximal cover frame 54, the support sleeve 50 and the endless belts 44. This is effective in preventing entry of foreign material into the clearance space between the support sleeve 50 and the elongated tube 16. Also, the cover flange 72 prevents entanglement of tissue of an inner wall of a body cavity in the course of turning the endless belts 44. The proximal cover frame 54 prevents the support sleeve 50 from shifting in a proximal direction.

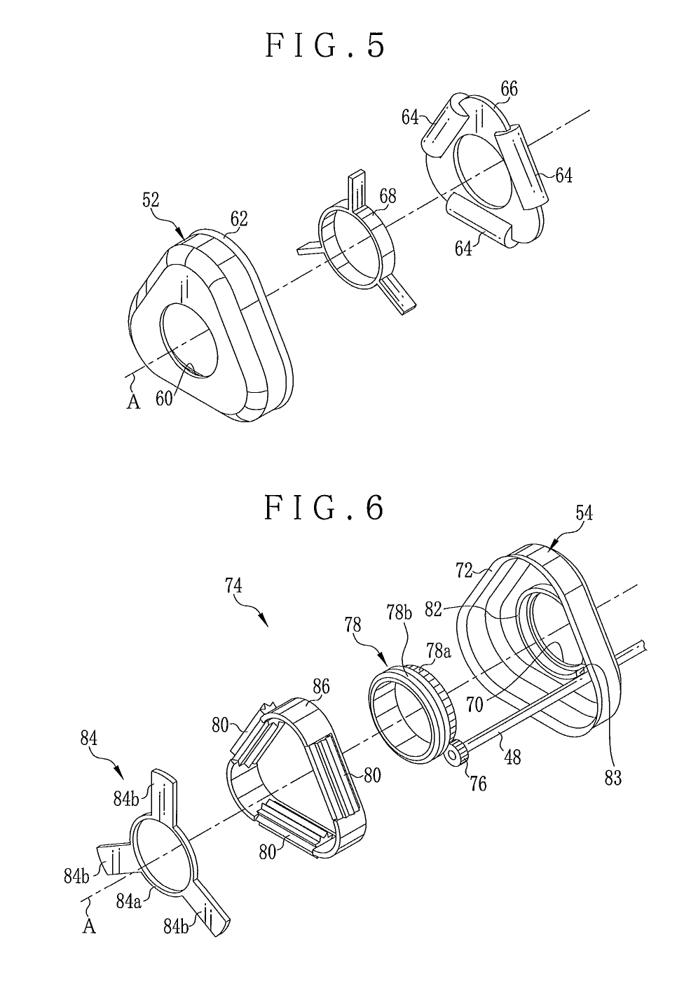

In FIG. 6, a gear set 74 or driving mechanism is contained in the proximal cover frame 54 for driving the endless belts 44. The gear set 74 includes an input gear 76, worm gear teeth 78b of a threaded sleeve 78 (worm gear), and a driving roller 80 or worm wheel or gear as a second regulating portion. The input gear 76 is coupled with a distal end of the control wire 48 and rotates together. The input gear 76 is supported on a distal surface of the proximal cover frame 54 in a rotatable manner.

The worm gear teeth 78b of the threaded sleeve 78 are disposed around the elongated tube 16, and rotatable about the axial direction A. A spur gear ring 78a is disposed at a proximal end of the worm gear teeth 78b, and meshed with the input gear 76. The worm gear teeth 78b are caused to rotate by the input gear 76. An inner surface of the threaded sleeve 78 is supported by the outside of the elongated tube 16. An annular ridge 82 is formed on the distal surface of the proximal cover frame 54. The cylindrical outer surface of the worm gear teeth 78b is supported by the annular ridge 82. The worm gear teeth 78b are prevented from shifting transversely to the axial direction A.

A cutout 83 is formed in the annular ridge 82 with a width corresponding to the input gear 76, which is meshed with the spur gear ring 78a through the cutout 83.

A retaining plate 84 is disposed on a distal side from the worm gear teeth 78b. The retaining plate 84 includes a center ring 84a and three radial projections 84b. The center ring 84a is disposed around the elongated tube 16. The radial projections 84b extend from the center ring 84a toward three corner portions of the proximal cover frame 54, the corner portions being disposed between the endless belts 44 by way of curved portions. Ends of the radial projections 84b are fixed on an inner surface of the proximal cover frame 54. The worm gear teeth 78b are disposed between the center ring 84a and the proximal cover frame 54, and prevented from shifting in the axial direction.

A roller holder 86 or polygonal ring supports the driving roller 80 (worm wheel as driving device) in a rotatable manner. Three of the driving roller 80 are arranged on the roller holder 86 at an interval of 120 degrees about the axial direction A of the elongated tube 16. The roller holder 86 is disposed between the proximal cover frame 54 and the retaining plate 84. A proximal end of the roller holder 86 is fixed to the proximal cover frame 54. A distal end of the roller holder 86 is fixed to each of the radial projections 84b.

The driving roller 80 (worm wheel as driving device) is meshed with the worm gear teeth 78b and caused to rotate by rotation of the worm gear teeth 78b. When the proximal cover frame 54 is mounted, the driving roller 80 is engaged with each of the endless belts 44, and rotates to turn around the endless belts 44. Also, the proximal cover frame 54 causes the driving roller 80 to regulate the endless belts 44, to prevent the support sleeve 50 from shifting in the proximal direction.

In short, force generated by the motor drive unit 42 in the propulsion assembly 14 is transmitted by the gear set 74 to the endless belts 44 for turn around, the gear set 74 including the input gear 76, the worm gear teeth 78b and the driving roller 80. The gear set 74 is contained in the proximal cover frame 54 and disposed on a proximal side from the endless belts 44, which are opposed to the peripheral surface of the elongated tube 16. No mechanical element is present between the lower belt run 89 of the endless belts 44 and the elongated tube 16. Thus, the diameter of the propulsion assembly 14 can be small so as to reduce physical stress to a patient.

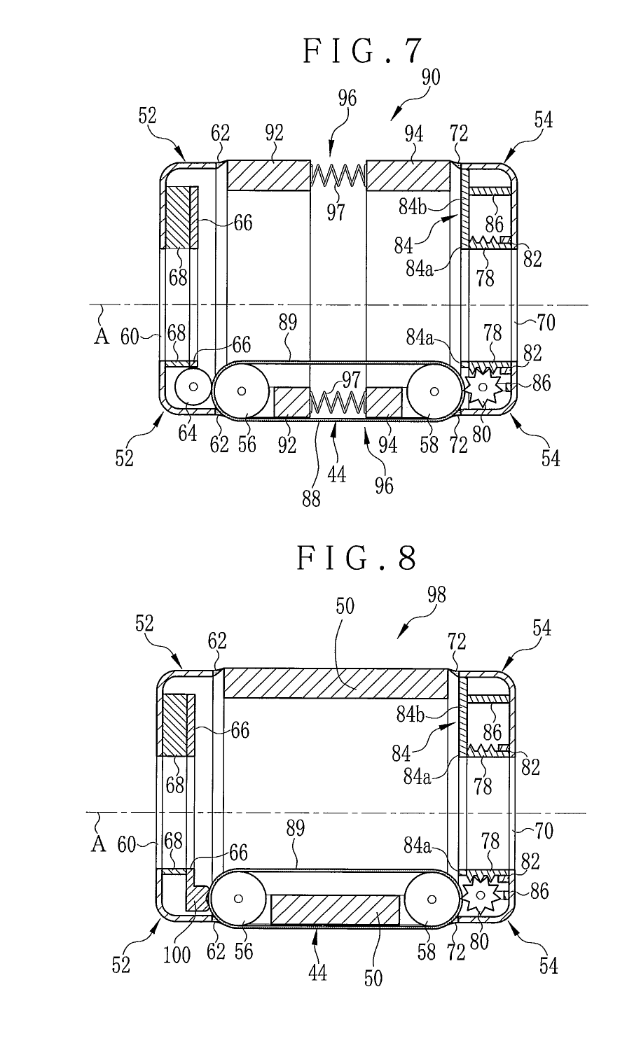

In FIG. 7, another preferred propulsion assembly 90 is illustrated. Each of the endless belts 44 is biased toward the idler roller 64 and the driving roller 80 (worm wheel as driving device). A support sleeve 96 in the propulsion assembly 90 includes a distal sleeve part 92 and a proximal sleeve part 94 or polygonal rings. A compression spring mechanism 97 or compression coil spring is disposed between the proximal and distal sleeve parts 92 and 94, biases the distal sleeve part 92 toward the idler roller 64 and the proximal sleeve part 94 toward the driving roller 80. Elements similar to those of the above embodiment are designated with identical reference numerals.

In FIG. 8, still another preferred propulsion assembly 98 is illustrated. A stationary pad portion 100 as a first regulating portion is used in place of the above-described idler roller, and contacts each of the endless belts 44 for preventing the same from shifting in the distal direction. Also, a gear set or driving mechanism may be disposed on a distal side from the endless belts 44. A stationary pad portion can be disposed on a proximal side, and prevent the endless belts 44 from shifting in the proximal direction.

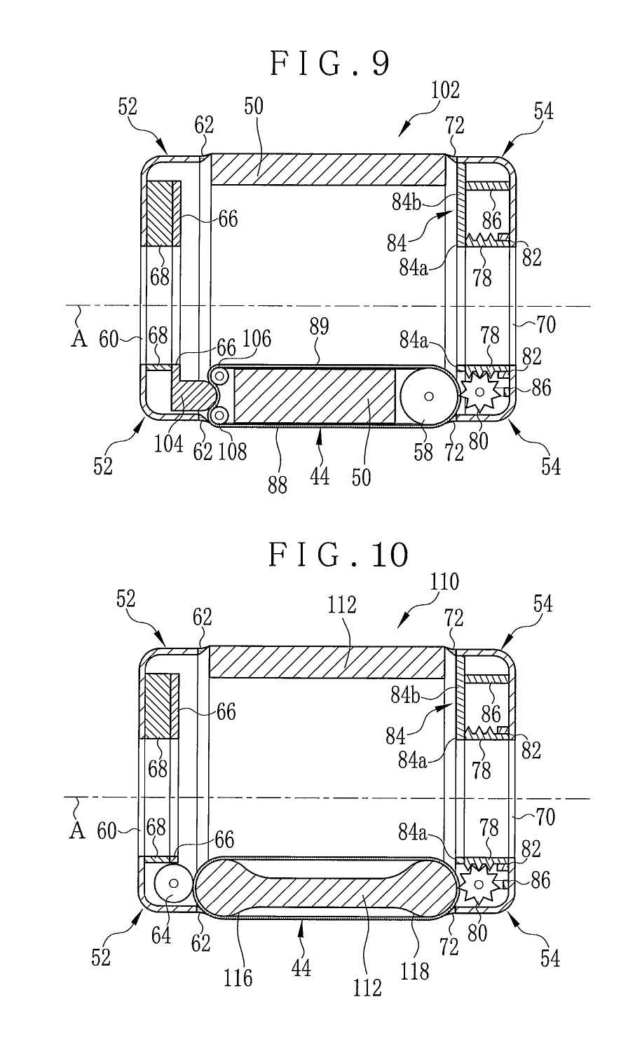

In FIG. 9, another preferred propulsion assembly 102 is illustrated, and includes two rotatable rolls 106 and 108 and a stationary pad portion 104 as a first regulating portion. The rotatable rolls 106 and 108 for the distal support roller are arranged so that the pad portion 104 is disposed between their positions. Furthermore, it is possible to use one distal roller and two pad portions. The distal roller may be disposed between their positions. Two idler rollers may be used with which the distal roller may be disposed between their positions. Also, two proximal rollers may be disposed. The worm wheel may be disposed between their positions. Furthermore, an idler roller can be disposed inside or outside the worm wheel. A single proximal roller may be disposed between positions of the idler roller and the worm wheel.

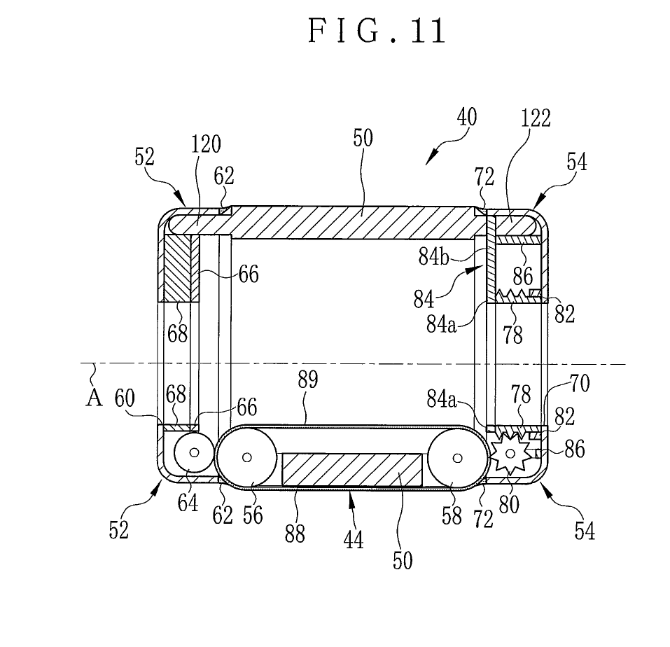

In FIG. 10, a further preferred propulsion assembly 110 is illustrated. A support sleeve 112 supports the endless belts 44 directly without proximal or distal rollers. The support sleeve 112 has first and second support surfaces 116 and 118 curved arcuately. The first support surface 116 is disposed on a distal side. The second support surface 118 is disposed on a proximal side. The support surfaces contact the endless belts 44 for reducing friction in a supported state.

In the above embodiments, the gear set is disposed on a proximal side from the endless belts. However, a gear set or driving mechanism can be disposed on a distal side from the endless belts. The control wire is disposed to extend to the distal cover frame. A gear set can have a structure inverted in the front-to-back orientation with respect to the axial direction, and can be contained in the distal cover frame. Also, two gear sets can be used and disposed on proximal and distal sides from the endless belts, and can drive the endless belts in synchronism.

In the above embodiment, the endless belts are three. However, the number of the endless belts may be one or two, or four or more.

In the above embodiment, the support sleeve is a specific type (floating type) which is prevented from shifting by the proximal and distal cover frames and the elongated tube. However, it is possible fixedly to secure the support sleeve to the elongated tube or to at least one of the proximal and distal cover frames. Preferably, engaging projections 120 and 122 or stays or coupling device of FIG. 11 can be disposed for connecting a first portion of the support sleeve to the elongated tube or to at least one of the proximal and distal cover frames 52 and 54, the first portion being positioned between the endless belts 44. The engaging projections 120 and 122 can position the support sleeve exactly. Thus, it is possible reliably to prevent the support sleeve from shifting finely. It is easier to mount the support sleeve to the elongated tube together with the proximal and distal cover frames 52 and 54 than to mount the support sleeve in a separate manner from the proximal and distal cover frames 52 and 54.

The engaging projections 120 and 122 project in parallel with one another from positions of vertices of the shape of the triangular prism. For example, those are three pins, bosses or the like, or three pairs. The engaging projections 120 and 122 are only received inside the proximal and distal cover frames 52 and 54 for positioning without coupling, but can be constructed for retention inside the proximal and distal cover frames 52 and 54 with firm coupling.

Note that the spacer 68 of the distal cover frame 52 and the retaining plate 84 of the proximal cover frame 54 are firmly retained on the periphery of the elongated tube 16 for the purpose of mounting the endless track device 40 on the elongated tube 16 inclusive of the support sleeve 50, 96, 112 and the proximal and distal cover frames 52 and 54. Thus, the support sleeve 50, 96, 112 is kept on the elongated tube 16 fixedly. However, there are various ideas of structures for fixedly mounting the endless track device 40 on the elongated tube 16. The structures can be used according to the purpose.

The lower belt run 89 of the endless belts 44 is directly opposed to the elongated tube 16, but runs between the proximal and distal support rollers 56 and 58 without influencing to the elongated tube 16 during the propulsion of the endless track device 40. In consideration of this, it is preferable to predetermine sizes and disposition of the proximal and distal support rollers 56 and 58 and the support sleeve to keep the lower belt run 89 movable freely from the elongated tube 16.

In the above embodiments, the endless track device has the endless belts. However, other endless track devices may be used. For example, an endless track device of U.S. Pat. Nos. 6,971,990 and 7,736,300 (corresponding to JP-A 2009-513250) in a toroidal shape can be used. It is possible to dispose the support sleeve in a center lumen of the endless track device and prevent the endless track device from shifting in the axial direction with the proximal and distal cover frames and the elongated tube.

In the above embodiments, the endoscope is for a medical use. However, an endoscope of the invention can be one for industrial use, a probe of an endoscope, or the like for various purposes.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A propulsion assembly for an endoscope having a section of an elongated tube for entry in a tube of a body cavity, comprising:
an endless track device for contacting a surface of said body cavity, and for propulsion by turning around in an axial direction of said elongated tube;
a support sleeve for receiving said elongated tube therein, and supporting said endless track device movably;
a distal cover frame, having a first through opening, for covering a distal side of said support sleeve in receiving said elongated tube in said first through opening;
a first regulating portion, contained in said distal cover frame, for contacting a distal end of said endless track device, to regulate said endless track device in said axial direction;
a proximal cover frame, having a second through opening, for covering a proximal side of said support sleeve in receiving said elongated tube in said second through opening;
a second regulating portion, contained in said proximal cover frame, for contacting a proximal end of said endless track device, to regulate said endless track device in said axial direction;
wherein at least one of said first and second regulating portions is a driving device, rotated by a drive source, for driving said endless track device.

2. A propulsion assembly as defined in claim 1, wherein said endless track device includes:
an upper run, disposed outside said support sleeve to extend in said axial direction, for contacting said surface of said body cavity;
a lower run, disposed inside said support sleeve to extend in said axial direction, and opposed to a peripheral surface of said elongated tube.

3. A propulsion assembly as defined in claim 2, wherein said lower run movably contacts said peripheral surface of said elongated tube.

4. A propulsion assembly as defined in claim 2, wherein said second regulating portion is said driving device.

5. A propulsion assembly as defined in claim 4, wherein said first regulating portion is an idler roller for rotating upon movement of said endless track device.

6. A propulsion assembly as defined in claim 2, wherein said endless track device includes plural endless belts arranged around said elongated tube to extend respectively in said axial direction.

7. A propulsion assembly as defined in claim 6, further comprising:
a distal support roller, secured to said support sleeve in a rotatable manner, opposed to said first regulating portion, for supporting each one of said endless belts movably;
a proximal support roller, secured to said support sleeve in a rotatable manner, opposed to said second regulating portion, for supporting each one of said endless belts movably.

8. A propulsion assembly as defined in claim 7, wherein said distal support roller includes:
a first rotatable roll disposed on an inner wall of said support sleeve in a rotatable manner;
a second rotatable roll disposed on an outer wall of said support sleeve in a rotatable manner;
said first regulating portion projects between said first and second rotatable rolls in a proximal direction.

9. A propulsion assembly as defined in claim 6, further comprising a coupling device for securing said support sleeve to at least one of said proximal and distal cover frames.

10. A propulsion assembly as defined in claim 9, wherein said coupling device includes a projection, formed to project from a portion of said support sleeve between said endless belts, for securing inside said proximal or distal cover frame.

11. A propulsion assembly as defined in claim 6, further comprising:
a first support surface, formed at a distal end of said support sleeve, curved arcuately, opposed to said first regulating portion, for supporting each one of said endless belts movably;
a second support surface, formed at a proximal end of said support sleeve, curved arcuately, opposed to said second regulating portion, for supporting each one of said endless belts movably.

12. A propulsion assembly as defined in claim 4, wherein said support sleeve includes:
a distal sleeve part disposed on a proximal side from said first regulating portion;
a proximal sleeve part disposed between said distal sleeve part and said second regulating portion;
a compression spring device, disposed between said proximal and distal sleeve parts, for biasing said distal sleeve part toward said first regulating portion, and biasing said proximal sleeve part toward said second regulating portion.

13. A propulsion assembly as defined in claim 4, further comprising:
a wire disposed to extend along said elongated tube, and rotated by said drive source;
an input gear secured to a distal end of said wire;
a sleeve having a worm gear and a spur gear ring supported on said proximal cover frame, disposed around said elongated tube, and caused to rotate by mesh with said input gear;
wherein said driving device is a worm wheel for rotating in mesh with said worm gear.

14. A propulsion assembly as defined in claim 4, wherein said first regulating portion is a pad portion formed to project from an inner surface of said distal cover frame toward said support sleeve.
